(19) United States
(12) Patent Application Publication
(10) Pub. No.: US 2025/0099166 A1
(43) Pub. Date: Mar. 27, 2025

(54) SPHERICAL (BALL) RADIO FREQUENCY ELECTRODE MULTIFUNCTIONAL FOR ABLATION OF NABOTHIAN CYST IN ENDOCERVICAL CANAL.

(71) Applicant: CESAR AUGUSTO PENA ACOSTA, Santo Domingo (DO)

(72) Inventor: CESAR AUGUSTO PENA ACOSTA, Santo Domingo (DO)

(21) Appl. No.: 18/195,935

(22) Filed: Sep. 27, 2023

(51) Int. Cl. A61B 18/14; A61B 1/303; A61B 18/00

(52) U.S. Cl. CPC A61B 18/1485; A61B 1/303; A61B 2018/00083; A61B 2018/00559; A61B 2018/00577; A61B 2018/1417; A61B 2218/002; A61B 2218/007

(57) ABSTRACT

The solution presented solves the problem of Nabothian cysts in the endocervix, using a multifunctional radiofrequency spherical electrode, which includes a sphere in the handle, internal conduit (suction-irrigation), thermal insulation, millimeter graduation, measurement fixation ring, which works as a stop, so that the electrode penetrates directly into the cyst directed by ultrasound, applying heat at an intensity (45-75) degrees Celsius and 20-30 seconds each application connected to an electrosurgical generator, also connected to a suction device for the mucous content of the same and irrigate through the canal, 0.9% saline solution, decreasing the intensity of the heat applied to the adjacent tissues. Treatment with healing creams, anti-inflammatories, antibiotics, antifungals and anesthetics may be applied. Managing to make the duct permeable, we will achieve improvement in dysmenorrhea, dyspareunia, fertility and endocervicitis.

### TECHNICAL FIELD OF INVENTION

The present utility model belongs to the technical field Scientific medicine—(Gynecological Surgery Area)

### PREVIOUS ART DESCRIPTION

In patent A61B 18/14 date of publication 25/05/00 in the World Intellectual Property Organization by Zeman, Laura, j. LEEP is a Loop Electrosurgical Excision Procedure (Loop Electrosurgical Excision Procedure). It is a treatment that prevents cervical cancer. A small electrical wire in the shape of a loop is used to remove the abnormal cells inside the cervix. LEEP surgery can be performed after abnormal cells are detected on a Pap smear, colposcopy, or biopsy.

You lie on a table as if you were having a Pap test. The doctor or nurse inserts a speculum into your vagina and opens it. This spreads the walls of the vagina apart so that the cervix can be seen.

When the doctor or nurse sees the cervix, he or she applies a local anesthetic to the area. He or she uses a small instrument with a wire loop that transmits an electrical current to eliminate the abnormal cells, user continue to cauterized the blood vessels in the area to prevent bleeding. They may also use a special paste called Monsel's solution to prevent bleeding. The cells are sent to a laboratory for analysis.

Loop electrosurgical excision (Leep) is applied using various loop electrodes and or spherical balls electrodes of 0.3 and 0.5 cms. With this existing technique, pathologies of viral etiology in the genital tract such as condyloma type warts and cervical dysplasia located in the exocervix and endocervix can be treated and eliminated by electrosurgery, eliminating portions of cone-shaped tissues by the loop (Leep). With the help of the ball electrode, only Nabothian cysts located in the exocervix can be eliminated.

### DETAILED DESCRIPTION OF THE INVENTION

The object of the present invention consists of a multifunctional radiofrequency electrode that can be used to eliminate or reduce Nabothian cysts located in the endocervical canal. It will be connected to an electrosurgical power generator and to an existing aspirator.

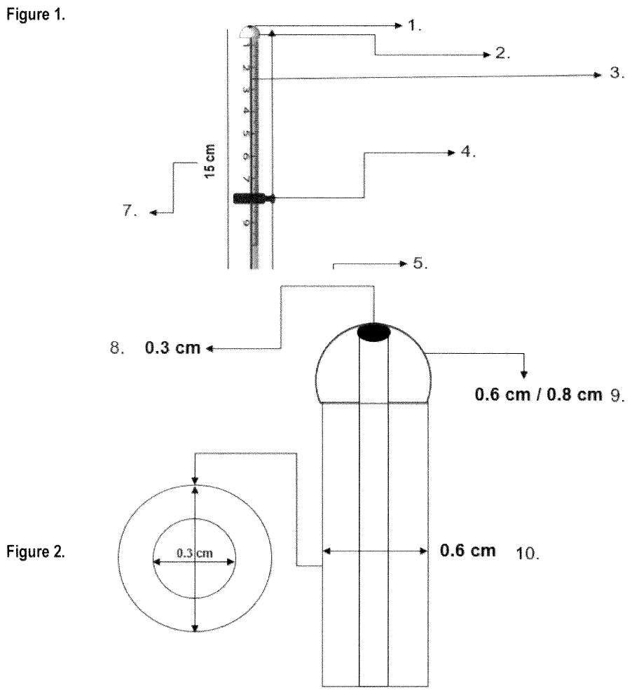

FIG. 1 shows a longitudinal view of the electrode.

1.1—Suction-irrigation duct: to extract mucous content in the endocervical canal and Nabothian cyst and irrigate the endocervical canal.

1.2—Ball or Sphere: apply heat to the Nabothian cyst (ablation-desiccation).

1.3—Measurements in cm and mm: To fix the distance between the Nabothian cyst in the endocervical canal and the external cervical os.

1.4—Measurement fixation ring: Holds the specified measurement to accurately direct the sphere or ball to the Nabothian cyst.

1.5—Thermal insulation: to avoid contact with adjacent tissues (Vulva-vaginal wall).

1.6—Electrode length: 20 centimeters.

1.7—Length of the suction-irrigation duct: 15 centimeters.

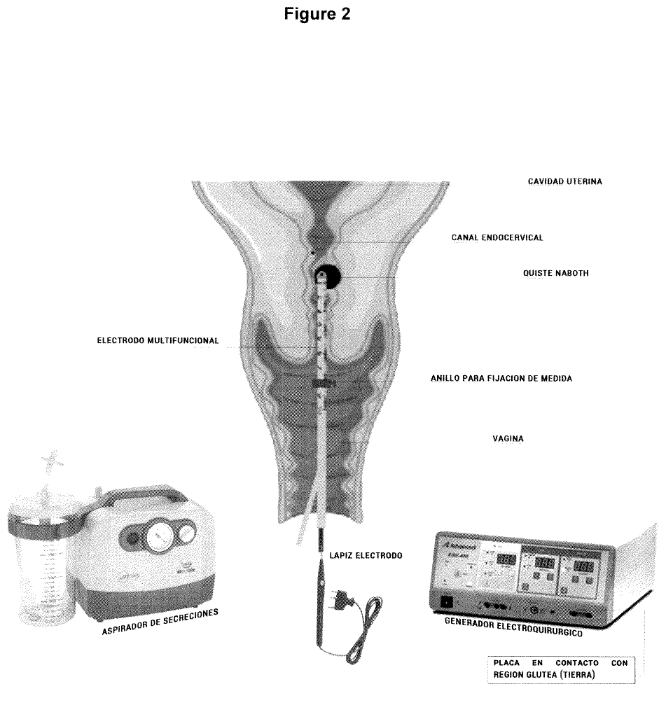

FIG. 2 shows a transverse view of the electrode.

2.8—Diameter of the suction-irrigation duct: 0.3 centimeters.

2.9—Diameter of the sphere or ball: 0.6 cm/0.8 cm.

2.10—Electrode diameter: 0.6 centimeters.

The multifunctional electrode has the following advantages over the existing well-known ball electrode.
1. Nabothian cysts located in the endocervical canal can be eliminated or reduced.
2. It would allow the removal of mucus from the cyst and endocervical canal.
3. It will allow irrigation (saline solution) in the area where the ablation treatment is applied.
4. It will allow the application of treatment with healing creams, analgesics, xylocaine gel, antibiotics, antifungal, and anti-inflammatory in the endocervix.

FIG. 1.

1. Multifunctional radiofrequency spherical electrode (ball) for Nabothian cyst ablation in endocervical canal, Its characterized by that it is composed of;
Internal suction-irrigation conduit according to claim number 1.

2. Measurements printed on the thermal insulator in cm and mm according to claim number 1.

3. Measurement fixing ring according to claim number 1.

4. It shall be compatible with the electrosurgical power generator (diathermic loop equipment), as well as the existing loop electrodes (Leep) as per claim number 1.

5. It shall be compatible with existing suction equipment as per claim number 1.

6. May be used to ablate Nabothian cysts in the endocervical canal as per claim number 1.

7. Electrode length: 15-20 centimeters according to claim number 1.

8. Length of the suction-irrigation conduit: 10-15 centimeters according to claim number 1. It could also have a mini-camera and LED system at the en of the spherical.

9. Diameter of the suction-irrigation duct: 0.3 centimeters in accordance with RECOMMENDATION NUMBER 1.

10. Electrode diameter: 0.6 centimeters in accordance with RECOMMENDATION NUMBER 1.

11. Diameter of the sphere or ball: 0.6 cm/0.8 cm according to REFERENCE NUMBER 1.
